(19) United States
(12) Patent Application Publication
(10) Pub. No.: US 2020/0352546 A1
(43) Pub. Date: Nov. 12, 2020

(54) SYSTEMS AND METHODS FOR CONTROLLING A SCREEN IN RESPONSE TO A WIRELESS ULTRASOUND SCANNER

(71) Applicant: Clarius Mobile Health Corp., Burnaby (CA)

(72) Inventors: Kris Dickie, Vancouver (CA); Laurent Pelissier, North Vancouver (CA)

(21) Appl. No.: 16/405,982

(22) Filed: May 7, 2019

(51) Int. Cl. A61B 8/00

(52) U.S. Cl. CPC A61B 8/4427; A61B 8/4472; A61B 8/14; A61B 8/54; A61B 8/565

(57) ABSTRACT

Mobile ultrasound scanners require a screen that acts as a display and user interface, yet there is often no convenient location for the screen. A mobile ultrasound scanner is therefore set up as a user interface to control a screen that is already in-situ. The ultrasound scanner wirelessly communicates with a separate interface module that controls a previously paired screen. When the scanner is in range of the interface module, the screen switches on. The interface module operates in an ultrasound scanning mode and optionally a user navigation mode, to which the screen display corresponds. The scanner operator can perform an ultrasound scan and provide user inputs without having to touch the screen. The scanner may have at least one physical button for operator input and/or a multi-axis accelerometer/gyroscope that detects gestures made using the scanner.

### TECHNICAL FIELD

This disclosure relates to medical ultrasound imaging systems. In particular, it relates to systems and methods for controlling a screen in response to a wireless ultrasound scanner.

### BACKGROUND

Ultrasound is a useful, non-invasive imaging technique capable of producing real time images. Ultrasound imaging has an advantage over X-ray imaging in that ultrasound imaging does not involve ionizing radiation.

When using traditional cart-based and laptop-based ultrasound systems, physicians and/or other ultrasound operators are typically required to operate the system from separate controls such as a keyboard. Some more modern mobile ultrasound scanners are app-based, and may be used with a screen-like add-on device that acts both as a display and a control device. Examples of these add-on devices include mobile phones, tablet or tablet computers, laptop and/or desktop computers.

While these app-based ultrasound systems have increased ease of use, they typically still require viewing on a separate add-on device. In some situations, the ultrasound operator cannot hold the add-on device because one hand is needed for the scanner and the other hand is needed for inserting a needle, for example. In mobile situations such as in an emergency department, or for regional anaesthesia procedures, users may not have a location to put the add-on device, and often the add-on device ends up being placed on the bed beside the patient. This also creates challenges because many times, there is not enough available space due to the presence of other medical equipment. In outdoor situations such as field EMS (emergency medical services), the add-on device may end up being placed on the patient themselves. In some cases, the add-on device has to be operated by someone else who is manipulate manipulating the scanner, e.g. for sterile procedures.

There is therefore a need for a display and UI (user interface) solution that frees physicians and/or other ultrasound operators from having to operate an ultrasound system from a separate touchscreen or keyboard, such as required when using cart-based and laptop-based units. In particular, there is a need for improved systems and methods for controlling a screen in response to a wireless ultrasound scanner.

The above background information is provided to reveal information believed by the applicant to be of possible relevance to the present invention. No admission is necessarily intended, nor should be construed, that any of the preceding information constitutes prior art against the present invention. The embodiments discussed herein may address and/or ameliorate one or more of the aforementioned drawbacks identified above. The foregoing examples of the related art and limitations related thereto are intended to be illustrative and not exclusive. Other limitations of the related art will become apparent to those of skill in the art upon a reading of the specification and a study of the drawings herein.

### BRIEF DESCRIPTION OF DRAWINGS

The following drawings illustrate embodiments of the invention and should not be construed as restricting the scope of the invention in any way.

FIG. 1 is a flowchart representing a high-level overview according to an embodiment of the present invention.

FIG. 2 is a schematic diagram of a system according to an embodiment of the present invention.

FIG. 3 is a sequence diagram representing a method involving UI navigation and ultrasound image control, according to an embodiment of the present invention.

FIG. 4 is a schematic diagram showing scanner gestures and corresponding effects on the display, according to an embodiment of the present invention.

FIG. 5 is a sequence diagram representing a method of using a scanner with a preset configuration to acquire ultrasound images according to an embodiment of the present invention.

FIG. 6 is a drawing of an operator performing an ultrasound scan while wearing augmented reality (AR) glasses, showing a display in the glasses superimposed on the patient.

### DETAILED DESCRIPTION

### A. Glossary

The term “conversion” refers to the construction of an ultrasound media, such as a still image or a video, from lines of ultrasound scan data representing echoes of ultrasound signals. “Conversion” may include performing ultrasound scan conversion on pre-scan-converted data (e.g., in polar coordinates) to generate a post-scan-converted image suitable for a particular display (e.g., to be in cartesian coordinates with the correct aspect ratio, etc.).

The term “operator” may refer to a person that is operating an ultrasound scanner (e.g., a clinician, medical personnel (including doctors, physicians, and/or veterinarians), a sonographer, ultrasound student, ultrasound instructor, ultrasonographer and/or ultrasound technician).

The term “module” can refer to any component in this invention and to any or all of the features of the invention without limitation. A module may be a software, firmware or hardware module, and may be located, for example, in the scanner, the interface, a network, a display device or a server.

The term “network” can include both a mobile network and data network without limiting the term's meaning, and includes the use of wireless (e.g. 2G, 3G, 4G, 5G, WiFi™, WiMAX™, Wireless USB (Universal Serial Bus), Zigbee™, Bluetooth™ and satellite), and/or hard wired connections such as local, internet, ADSL (Asymmetrical Digital Subscriber Line), DSL (Digital Subscriber Line), cable modem, T1, T3, fiber-optic, dial-up modem, television cable, and may include connections to flash memory data cards and/or USB memory sticks where appropriate. A network could also mean dedicated connections between computing devices and electronic components, such as buses for intra-chip communications.

The term “processor” can refer to any electronic circuit or group of circuits that perform calculations, and may include, for example, single or multicore processors, multiple processors, an ASIC (Application Specific Integrated Circuit), and dedicated circuits implemented, for example, on a reconfigurable device such as an FPGA (Field Programmable Gate Array). A processor may perform the steps in the flowcharts and sequence diagrams, whether they are explicitly described as being executed by the processor or whether the execution thereby is implicit due to the steps being described as performed by code or a module. The processor, if comprised of multiple processors, may be located together or geographically separate from each other. The term includes virtual processors and machine instances as in cloud computing or local virtualization, which are ultimately grounded in physical processors.

The term “system” when used herein, and not otherwise qualified, refers to a system for establishing a communication link between a wireless ultrasound scanner and an interface that controls a screen, in which the scanner provides signals to the interface for controlling the display on the screen and/or representing navigational and other inputs made using the scanner, the system being the subject of the present invention.

The term “ultrasound media” herein refers to an ultrasound video, ultrasound image feed, and/or a still ultrasound image. A frame of an ultrasound video may be referred to as a “still”. An ultrasound video may be generated live or pre-recorded, or may be artificially generated, e.g. for training purposes.

### B. Exemplary Embodiments

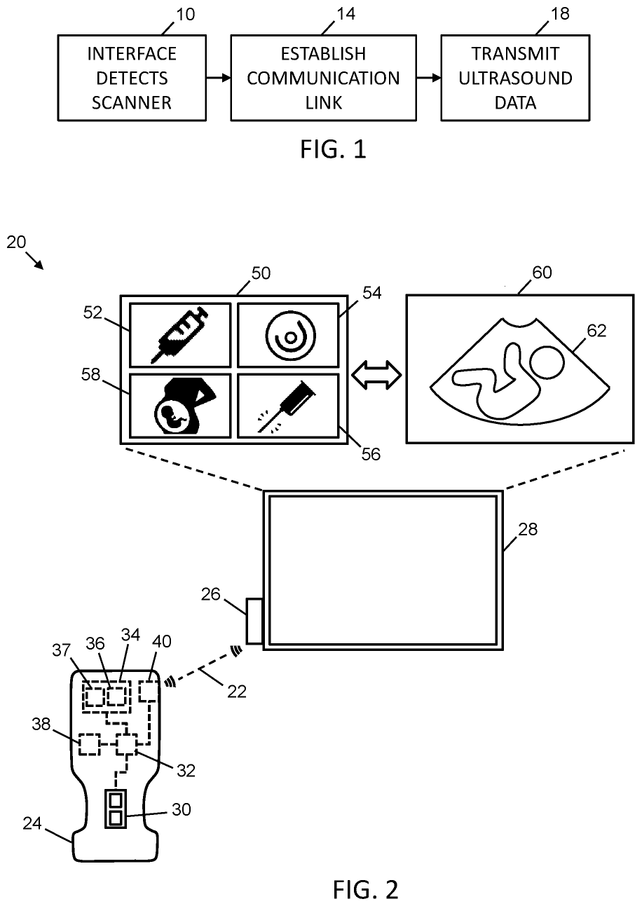

Referring to FIG. 1, a flowchart provides a high-level overview of the main functions undertaken in an exemplary embodiment of the invention. In step 10, an interface detects the presence of a wireless ultrasound scanner that is in its vicinity, which may be a result of the scanner broadcasting its identity. As used herein, an “interface” may be any suitable electronic components that are configured to perform the acts described below as being performed by the interface. In various embodiments, the interface may form part of a device that incorporates a screen (e.g., a smartphone, tablet computer, or other smart device), and/or the interface may be a device that is separate from the screen (e.g., an independent electronic device with a port for connecting to a wall-mounted display).

In step 14, the interface and the scanner establish a wireless communication link. In various embodiments, the interface and the scanner may have been previously paired and/or otherwise set up to automatically establish communication with each other when in range of each other. In step 18, the scanner may transmit ultrasound data to the interface, which may convert the data for display on a screen. The scanner may also transmit one or more parameters that instruct the interface how to convert the data.

Referring to FIG. 2, an exemplary system 20 is shown for establishing a communication link 22 between a wireless ultrasound scanner 24 (also referred to herein as “scanner” for brevity) and an interface 26 that is communicably coupled to a screen 28. Communication link 22 may use any suitable wireless network connection.

The scanner 24 may include zero or more physical switches 30. In various embodiments, switches 30 may be provided in the form one or more buttons. The switches 30 may be used by an operator of the scanner 24 to provide one or more inputs to the system 20. Such inputs may be, for example, commands to navigate between selectable options displayed on the screen 28, selection of an option displayed on the screen 28, the input of alphanumeric information (e.g., relating to a patient), the adjustment of the gain of the displayed ultrasound media, the adjustment of the depth of the displayed ultrasound media, freezing a video media of an ultrasound scan, and/or the change of the mode of the system 20.

The screen 28 may be, for example, a television screen, a wall-mounted display, a laptop screen, a desktop screen, a tablet screen, a mobile phone screen, a watch screen and/or wearable smart/AR glasses.

The switches 30 may be connected to a processor 32, which may be connected to a non-transitory computer readable memory 34 storing computer readable instructions 36, which, when executed by the processor 32, cause the scanner 24 to provide one or more of the functions of the system 20. Such functions may be, for example, the detection of an interface 26, the establishment of a communication link 22 with the interface 26, the transmission of ultrasound data to the interface 26, and the detection of operator inputs to the scanner 24.

Also stored in the computer readable memory 34 may be computer readable data 37, which may be used by the processor 32 in conjunction with the computer readable instructions 36 to provide the functions of the system 20. Computer readable data 37 may include, for example, configuration settings for the scanner 24, such as a preset parameter that instructs the interface 26 how to convert the ultrasound data that is transmitted by the scanner 24 to the interface 26. For example, preset data may include ultrasound imaging parameters such as frequency, depth, Time Gain Compensation (TGC), and/or beamformer parameters (such as the number of focal zones and/or focus position) that are tuned/optimized for a particular type of imaging. For example, there may be image presets for obstetrics/gynecology (OB/GYN), abdomen, cardiac, small parts, vascular, and/or other medical applications. Configuration settings may include any other data that is specific to the way that the scanner 24 operates.

In some embodiments, the processor 32 may be connected to a multi-axis motion sensor 38, for example a combination of a 3-axis accelerometer and 3-axis gyroscope, which detects movements of the scanner 24 and sends signals representing such movements to the processor 32. In various embodiments, the motion sensor 38 may be provided in the form of an inertial measurement unit (IMU).

The scanner 24 may include a communications module 40 connected to the processor 32. The communications module 40 may wirelessly transmit signals to and receives signals from the interface 26. The protocol used for communications between the scanner 24 and the interface 26 may be WiFi™ or Bluetooth™, for example, or any other suitable two-way radio communications protocol. The scanner 24 may operate as a WiFi™ hotspot, for example.

The interface 26 may be a device that is a separate from the screen 28, or in other embodiments the interface 26 may be incorporated integrally into the same device that hosts the screen 28. The functions of the interface 26 are, for example, to listen for the scanner 24, establish a communication link 22 with the scanner 24, receive data, including configuration settings and imaging parameters from the scanner 24, convert ultrasound data that is received from the scanner 24 into an ultrasound media that is displayable on the screen 28, and switch from one mode to another in response to a mode change in the scanner 24. When working together, the scanner 24 and interface 26 may operate multiple modes. For example, one mode may be a scan mode, in which ultrasound data is obtained by the scanner 24 and transmitted to the interface 26 for conversion and display on the display 28. Another mode may be a navigation mode in which the interface 26 receives, for example, navigation inputs, option selections and alphanumeric data input from the scanner 24.

The interface 26 may output signals to the screen 28 resulting in the display on the screen 28 of a navigation UI 50, which may include one or more selectable icons 52, 54, 56, 58, for example. In this example, icon 52 represents preset options for anesthesiology, icon 54 represents preset options for breast scanning, icon 56 represents preset options for intervention and icon 58 represents preset options for OB/GYN. As noted, each preset option may have corresponding settings and imaging parameters (e.g., depth and gain settings) that are optimized for the type of ultrasound scan that is to be performed. These settings and the presets to which they correspond may be stored as part of the computer readable data 37. In other embodiments, other icons may be provided, including icons that enable different types of operator data input at the scanner 24.

The screen 28 may be considered a touchless UI, as the operator does not need to touch the screen 28 either to switch it from its standby or hibernation state or to navigate its various UI.

The interface 26 may also receive signals from the scanner 24 that represent changes in the mode of the scanner 24, for example a change between a navigation mode and a scan mode. The interface 26 operates in a mode that follows the mode of the scanner 24, such that the scanner 24 and interface 26 both operate simultaneously in the navigation mode or they both operate simultaneously in the scan mode.

When the interface 26 is operating in the scan mode, it causes the display on the screen 28 of a scanning UI 60 which shows, for example, an ultrasound image feed of a fetus 62.

Interface 26 may include electronic circuitry, for example a processor and computer readable memory storing computer readable instructions which, when executed by the processor, result in the interface 26 performing one or more of the functions described herein.

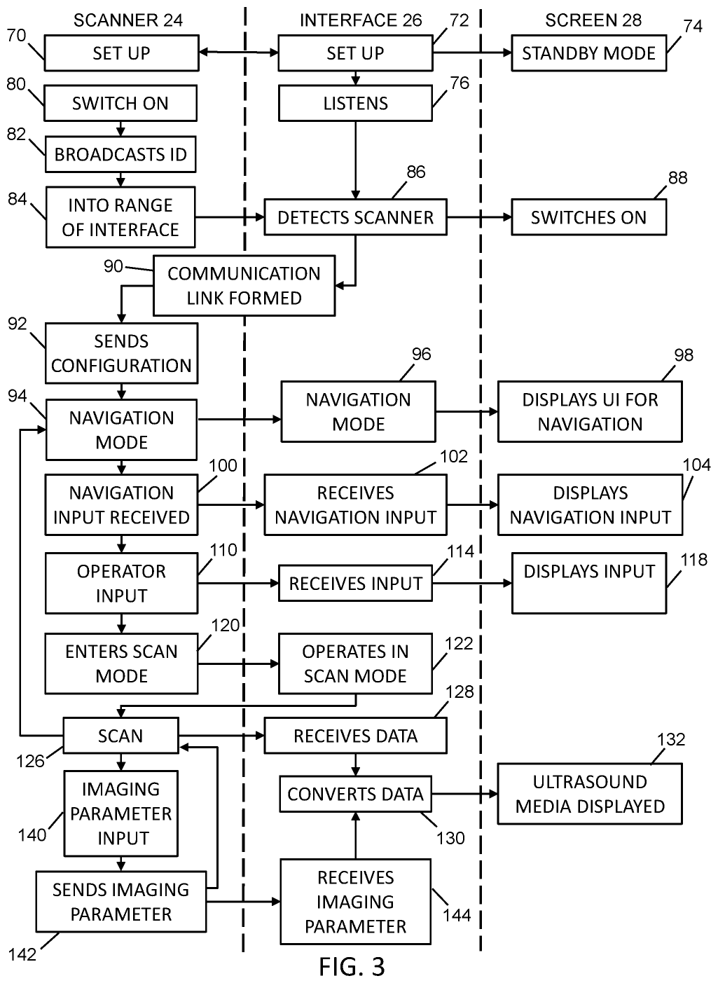

Referring to FIG. 3, a flowchart of an exemplary method is shown that is performed by the scanner 24, interface 26 and screen 28, where the scanner 24 and interface 26 operate in navigation and scan modes.

In step 70, the scanner 24 is set up in conjunction with the interface 26 being set up in step 72. During the set up steps 70, 72, the scanner 24 and the interface 26 may be paired so that they may later recognize each other when they are within range of each other. The range may depend on the signal strength of the radio signals that the scanner 24 and the interface 26 are able to emit. Depending on the application, the distance within which the scanner 24 and interface 26 recognize they are within range may differ. For example, if the interface 26 and display 28 is provided in the form of smart/AR glasses, then it may be desired for the distance to be shorter. However, if the interface 26 and display 28 is provided on a wall-mounted display, then the distance may be desired to be greater. In various embodiments, the distance for the interface 26 to detect when the scanner 24 is in range may be, for example, between 2 meters −50 meters of the interface 26.

After the interface 26 has been set up, the screen 28 may be instructed by the interface 26 to enter the standby or hibernation mode, in step 74. Whenever the screen 28 is switched off, e.g. as a result of a person switching it off or a power cut, it may automatically enter the standby mode when it is switched on again. Also, after the interface 26 has been set up, the interface 26 may then enter a listening mode, in step 76. In the listening mode, the interface 26 may listen for signals that are broadcast by other electronic wireless devices that are within its range of detection. In various embodiments, in listening mode, the interface 26 may be configured to periodically send a beacon message/frame (as is available in various wireless communication protocols such as Wi-Fi™ 802.15.4, Zigbee™ and/or Bluetooth™) to continually announce its presence to nearby scanners 24.

In step 80, the scanner 24 may be switched on, for example by an operator who intends to perform an ultrasound scan. After switching on, the scanner 24 may broadcast its identification (ID) signal in step 82 so that other electronic devices within its range can detect it. In step 84, the scanner 24 may be brought into the range of the interface 26, at which point the interface 26 can detect the scanner 24 in step 86. As a result of detecting the scanner 24, the interface 26 may instruct the screen 28 to switch on, in step 88. Also, as a result of the interface 26 detecting the scanner 24 in step 86, the interface 26 and scanner 24 may establish a communication link 22 in step 90. Both the scanner 24 and the interface 26 may participate in the establishment of the communication link 22. The establishment of the communication link 22 may involve, for example, the interface 26 transmitting a request to the scanner 24 to establish a communication link 22, and the scanner 24 responding to the request by accepting the request. Additional handshake messages and/or other communication setup messages may be exchanged to establish the communication link 22.

In step 92, the scanner 24 may send any necessary configuration settings to the interface 26. The configuration settings may define how the transducer elements of the scanner 24 are arranged (e.g., linearly, curved, or some other fashion), and or the how transducer elements are activated (e.g., sequentially or in a phased manner). Configuration settings may also include one or more imaging parameters, which define, at least in part, how the interface should convert the ultrasound data captured by the scanner 24. For example, the imaging parameters may define the default depth, frequency, TGC, beamforming parameters and/or gain settings. The configuration setting may further include any other characteristics of the scanner 24, including any other information that permits scan conversion to be performed at the interface 26.

In step 94, the scanner 24 enters the navigation mode, and as a result, may send a signal to the interface 26 to cause it to also enter the navigation mode, in step 96. In turn the interface 26 may send signals to the screen 28 to cause it to display the UI for navigation, in step 98 (e.g., the navigation UI 50 shown in FIG. 2). In various embodiments, the particular elements of the navigation UI 50 displayed in the navigation mode may be included in the configuration settings.

In step 100, the scanner 24 may receive a navigation input, such as an input for moving a point of focus on the navigation UI 50 (e.g. cursor or highlight) from one location to another, or from one icon to another. In step 102, the interface 26 may receive the navigation input from the scanner 24 and then commands the screen 28 to update the display of the navigation UI 50 in accordance with the navigation input, in step 104.

In step 110, the scanner 24 may receive an operator input, which may be, for example, the input of alphanumeric information or the selection of an option highlighted on the screen 28. In step 114, the interface may receive the operator input and may command the screen 28 to display the operator input or to otherwise alter the navigation UI 50 in response to the interface 26 receiving the operator input.

In step 120, the scanner 24 may enter the scan mode, for example as a result of the operator of the scanner 24 pressing a button 30 on the scanner 24 (as shown in FIG. 2) or making a gesture with the scanner 24, or otherwise selecting an option highlighted on the screen 28 that leads to a change in mode of the scanner 24 and interface 26 from the navigation mode to the scan mode. In response to the scanner 24 entering the scan mode, the interface 26 may correspondingly operate in the scan mode in step 122.

The scanner 24 may then be used to perform an ultrasound scan in step 126. During the scanning procedure, the scanner 24 may send ultrasound data to the interface 26, which is received by the interface 26 in step 128. The interface 26 then converts this data in step 130 to form an ultrasound media signal which is sent to the screen 28, on which the ultrasound media is displayed in step 132.

During the scanning procedure, the operator may input an imaging parameter to the scanner 24, in step 140. This parameter may represent a change in the depth or the gain, for example, and may be input to the scanner 24 using one or more of the buttons 30 on the scanner 24 (as shown in FIG. 2). Where the ultrasound media is a video, this parameter may be a command to freeze the video, for example. In response, the scanner 24 may send image control signals corresponding to the imaging parameter to the interface 26, in step 142. In step 144, the interface 26 may receive the imaging parameter and may then uses it in step 130 to convert the ultrasound data received (step 128) as the scanning procedure continues (step 126). The ultrasound media, adjusted by the imaging parameter, may be displayed on the screen in step 132. At any point in the scan, during step 126, the operator may switch the scanner 24 from the scan mode to the navigation mode, at which point the process may revert to step 94. The scanner 24 may be switched from the scan mode to the navigation mode by, for example, activating a switch 30 on the scanner 24 and/or by making a gesture with the scanner 24 that is distinctly different from a scanner movement that is normal during an ultrasound scanning procedure.

The scanner 24 may be switched off by the operator at any time, for example by entering the navigation mode and then selecting a switch-off option, by removing a removable battery, and/or by activating a power switch. In response, the interface 26 may enters the listening mode (step 76) and the screen 28 may enters the standby mode (step 74). Additionally or alternatively, the interface 26 may automatically switch off after a predetermined period of inactivity.

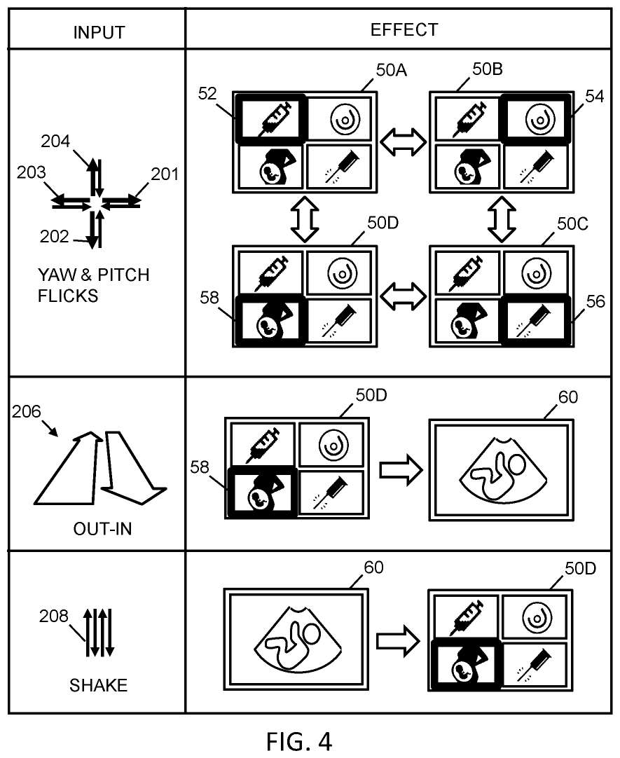

Referring to FIG. 4, exemplary inputs provided by way of scanner gestures and their corresponding effects on the navigation UI 50 are shown when the scanner 24 and the interface 26 are operating in the navigation mode. When the scanner 24 detects a gesture, it may transmit a control signal to the interface 26, which in turn updates the display of the navigation UI 50 in accordance with the control signal for the gesture inputted using scanner 24.

In the first row, the input gestures shown are flicks of the scanner 24 in four different directions (e.g., when pointing the distal/transducer element end of the scanner 24 away from the operator). These flicks involve a rapid movement in one direction (illustrated with an arrow in the desired direction with a heavier line weight) and then more slowly relaxing the wrist back to the starting position (illustrated with a corresponding arrow in the opposite direction in lighter line weight).

For example, right-yaw-flick 201 represents the operator flicking the scanner 24 rapidly to the right with a bend in the wrist, and then more slowly relaxing the wrist back to the starting position. This may cause the navigation UI to change from 50A, in which the anesthesiology icon 52 is highlighted, to 50B in which the breast icon 54 is highlighted. It may also cause the navigation UI to change from 50D to 50C.

Down-pitch-flick 202 represents the operator flicking the scanner 24 rapidly downwards with a bend in the wrist, and then more slowly relaxing the wrist back to the starting position. This may cause the navigation UI to change from 50B, in which the breast icon 54 is highlighted, to 50C, in which the intervention icon 56 is highlighted. It may also cause the navigation UI to change from 50A to 50D.

Left-yaw-flick 203 represents the operator flicking the scanner 24 rapidly to the left with a bend in the wrist, and then more slowly relaxing the wrist back to the starting position. This causes the navigation UI to change from 50C, in which the intervention icon 56 is highlighted to 50D, in which the OB/GYN icon 58 is highlighted. It may also cause the UI to change from 50B to 50A.

Up-pitch-flick 204 represents the operator flicking the scanner 24 rapidly upwards with a bend in the wrist, and then more slowly relaxing the wrist back to the starting position. This causes the navigation UI to change from 50D, in which the OB/GYN icon 58 is highlighted, to 50A, in which the anesthesiology icon 52 is highlighted. It may also cause the UI to change from 50C to 50B.

The middle row shows an extended out-in gesture 206, in which the scanner 24 is moved steadily away from the operator and then steadily back again, for example, out towards the screen 28 and then back from the screen 28. This gesture is markedly different from the flick gestures in the first row. The result of the out-in gesture 206 is to select a highlighted option, for example here the OB/GYN icon 58 is highlighted and the corresponding OB/GYN option is selected when the out-in gesture 206 is detected by the scanner 24. The result is that the screen 28 changes from the navigation UI 50D in the navigation mode to the scanning UI 60 in the scan mode, in which an ultrasound media can be displayed.

The bottom row shows a shake gesture 208, which may be, for example, a double up and down movement of the scanner 24. The result of the shake gesture 208, which is markedly different from any movement that is normally made during a scanning procedure, may be to switch the scanner 24 from the scan mode to the navigation mode. In this case, the screen 28 may change from displaying the ultrasound media in the scanning UI 60, to the navigation UI 50D.

The scanner 24 may be configured to recognize only the shake gesture 208 and not the flick gestures 201-204 or out-in gesture 206 while it is in the scan mode. This may reduce the likelihood of the movements of the scanner 24 during the ultrasound scan procedure being misinterpreted as gestures for navigation.

In other embodiments, different gestures may be used to navigate the navigation UI 50, and/or to switch between the navigation and the scan modes of the system 20.

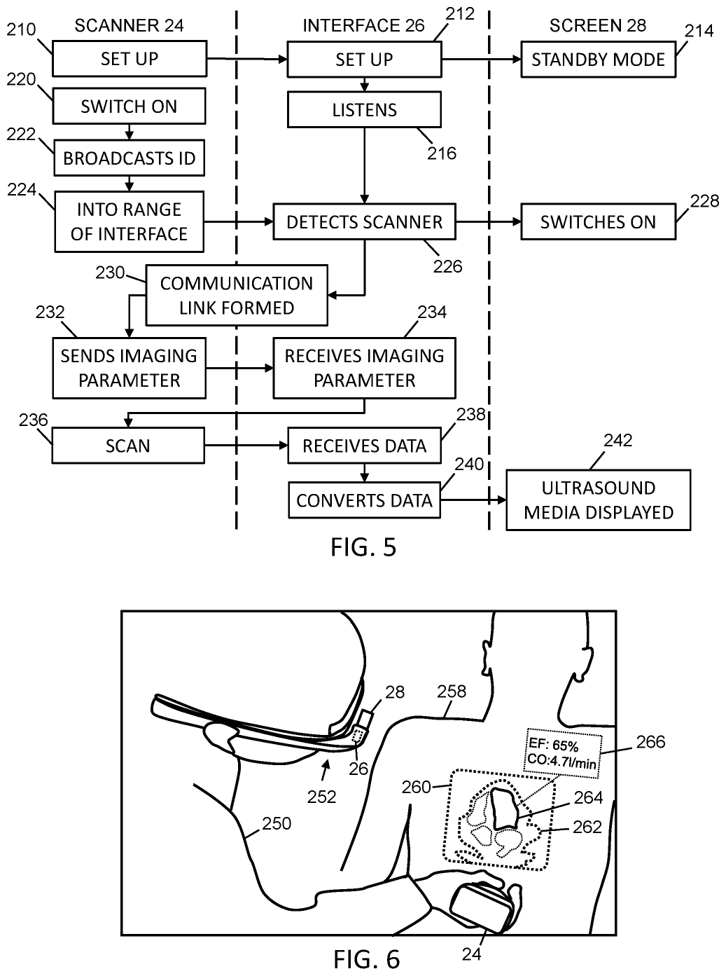

Referring to FIG. 5, a flowchart of an exemplary method is shown that is performed by the scanner 24, interface 26 and screen 28, where the scanner 24 and interface 26 operate in only a scan mode, and a number of imaging parameters (e.g., depth and gain settings) are automatically provided without ability for configuration by the operator. Such an embodiment may be desirable in the case where the scanner 24 is only used for one particular type of ultrasound scanning procedure, in which the depth, gain, or other settings do not need to be adjusted. An example application where this may be desirable is vascular access (e.g., where ultrasound imaging can be used to locate veins for administration of intravenous fluids or blood sampling). This application may be particularly suitable for the application of FIG. 5 because the tissue being imaged is relatively superficial, and deeper imaging is not required.

Acts 210-230 of FIG. 5 may generally be performed in a manner similar to how acts 70-90 were performed in FIG. 3, as discussed above.

Once a communication link is established, in step 232, the scanner 24 may send any necessary imaging parameters to the interface 26. For example, this may include default depth and frequency parameters. Step 232 may also involve configuration settings being transmitted to the interface 26. The imaging parameters and configuration settings transmitted at act 232 may be analogous to those discussed above as being transmitted in acts 92 and 142 of FIG. 3, as discussed above. In step 234, the interface 26 receives this information.

In step 236, the scanner 24 is used to perform an ultrasound scan. During the scanning procedure, the scanner 24 may send ultrasound data to the interface 26, which is received by the interface 26 in step 238. The interface 26 may then convert this data in step 240, using the previously received imaging parameters and configuration settings, to form an ultrasound media signal which is sent to the screen 28. At act 242, the ultrasound media can be displayed.

A number of acts in FIG. 5 correspond to acts in FIG. 3 discussed above. However, the method of FIG. 5 provides a simpler mode of operation (e.g., without a navigation mode to switch between different types of ultrasound imaging applications). Removing the potential complexity involved with changing modes, presets and imaging parameters may make ultrasound imaging easier and less intimating to use if the operator only has a single medical application (e.g., vascular access) which they are using ultrasound imaging for. Decreasing complexity in the software instructions providing this functionality may also improve initial boot-up time and imaging user responsiveness for users who do not need access to the removed features.

Referring to FIG. 6, shown there is an example embodiment where the interface 26 and screen 28 are provided together in the form of a pair of smart/AR glasses 252. As illustrated, an operator 250 wearing the smart/AR glasses 252 is performing an ultrasound scan with scanner 24 on a patient 258. The display 28 integrated with the smart/AR glasses 252 may show a virtual screen 260 (for illustrative purposes, shown in dotted outline as superimposed on the patient 258), which is visible to the operator 250. The virtual screen 260 may display the ultrasound media 262, of which part 264 of it representing a portion of the anatomy of the patient is highlighted by the AR glasses 252. The AR glasses 252 may also display an annotation 266 that provides information about the highlighted part 264 of the ultrasound media 262. In the illustrated example, the annotation is for the cardiac measurements ejection fraction (EF) and cardiac output (CO), and the annotation reads “EF: 65% CO:4.7 L/min”.

### C. Variations

Variations of the embodiments described herein are possible, examples of which are provided below.

When in the scan mode and the ultrasound media has been frozen, the scanner 24 may be used to make measurements on the frozen image. For example, the accelerometer and gyroscope data may be used to direct a pointer on a frozen ultrasound image to connect dots for the purposes of marking a measurement, or noting a spot on an ultrasound image.

Additionally or alternatively, other wireless communication protocols may be used provided that they can support sufficient bandwidth for transmitting the ultrasound data.

Additionally or alternatively, mechanisms other than the buttons 30 may be used to provide operator input. For example, these mechanisms may include using voice control (e.g., using voice assistants such as Apple™ Siri™, Google Assistant™ and/or Amazon™ Alexa™). For example, the scanner 24 may be fitted with a microphone so that voice commands can be input. Additionally or alternatively, a foot pedal may be connected to the scanner 24, either wired or wirelessly, so that the functions described for the buttons 30 can be incorporated into the foot pedal.

In various embodiments, the screen 28 may be part of another medical system that is already on-site where the ultrasound scan is to take place, and the interface 26 may be configured to commandeer the screen 28 upon the scanner 24 entering the range of the interface when switched on.

Embodiments of the invention may be implemented using specifically designed hardware, configurable hardware, programmable data processors configured by the provision of software (which may optionally include ‘firmware’) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”) and the like. Examples of configurable hardware are: one or more programmable logic devices such as programmable array logic (“PALs”), programmable logic arrays (“PLAs”) and field programmable gate arrays (“FPGAs”). Examples of programmable data processors are: microprocessors, digital signal processors (“DSPs”), embedded processors, graphics processors, math co-processors, general purpose computers, server computers, cloud computers, main computers, computer workstations, and the like. For example, one or more data processors in a control circuit for a device may implement methods as described herein by executing software instructions in a program memory accessible to the processors.

While processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times.

The embodiments may also be provided in the form of a program product. The program product may include any transitory or non-transitory medium which carries a set of computer-readable instructions which, when executed by a data processor, cause the data processor to execute a method of the invention. Program products according to the invention may be in any of a wide variety of forms. The program product may include, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

Where a component (e.g. software, processor, support assembly, valve device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

In some embodiments, the components of the systems and apparatuses may be integrated or separated. Moreover, the operations of the systems and apparatuses disclosed herein may be performed by more, fewer, or other components and the methods described may include more, fewer, or other steps. In other instances, well known elements have not been shown or described in detail and repetitions of steps and features have been omitted to avoid unnecessarily obscuring the invention. Screen shots may show more or less than the examples given herein. Accordingly, the specification is to be regarded in an illustrative, rather than a restrictive, sense.

It is therefore intended that the appended claims and claims hereafter introduced are interpreted to include all such modifications, permutations, additions, omissions and sub-combinations as may reasonably be inferred. The scope of the claims should not be limited by the embodiments set forth in the examples but should be given the broadest interpretation consistent with the description as a whole.

### D. Interpretation of Terms

Unless the context clearly requires otherwise, throughout the description and the claims, the following applies:

In general, unless otherwise indicated, singular elements may be in the plural and vice versa with no loss of generality. The use of the masculine can refer to masculine, feminine or both.

The terms “comprise”, “comprising” and the like are to be construed in an inclusive sense, as opposed to an exclusive or exhaustive sense, that is to say, in the sense of “including, but not limited to”.

The terms “connected”, “coupled”, or any variant thereof, means any connection or coupling, either direct or indirect, between two or more elements; the coupling or connection between the elements can be physical, logical, or a combination thereof.

The words “herein,” “above,” “below” and words of similar import, when used in this application, refer to this application as a whole and not to any particular portions of this application.

The word “or” in reference to a list of two or more items covers all of the following interpretations of the word: any of the items in the list, all of the items in the list and any combination of the items in the list.

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present) depend on the specific orientation of the apparatus described and illustrated. The subject matter described herein may assume various alternative orientations. Accordingly, these directional terms are not strictly defined and should not be interpreted narrowly.

To aid the Patent Office and any readers of any patent issued on this application in interpreting the claims appended hereto, applicant wishes to note that they do not intend any of the appended claims or claim elements to invoke 35 U.S.C. 112(f) unless the words “means for” or “step for” are explicitly used in the particular claim.

### E. Claim Support

In a first broad aspect of the present disclosure, there is provided a method for establishing a wireless communication link between an ultrasound scanner and an interface that controls a screen, the method comprising: broadcasting, by the ultrasound scanner, an identification signal; detecting, by the ultrasound scanner, a request from the interface to establish a communication link with the ultrasound scanner; establishing, by the ultrasound scanner, the communication link; sending, by the ultrasound scanner to the interface, data from an ultrasound scan; and sending, by the ultrasound scanner to the interface, a parameter that is used by the interface to convert the data into an ultrasound media for display on the screen.

Some embodiments may include: the ultrasound scanner operating as a hotspot to broadcast the identification signal and establish the communication link; or the ultrasound scanner using a Bluetoot™ communication protocol to broadcast the identification signal and establish the communication link.

Some embodiments may be performed by the ultrasound scanner without human intervention, after the ultrasound scanner is switched on.

Some embodiments may comprise: detecting, by the ultrasound scanner, a mode of the ultrasound scanner, wherein the mode is at least one of a navigation mode or a scanning mode; and sending, by the ultrasound scanner to the interface, a signal indicative of the mode; wherein the signal instructs the interface to operate in a mode that corresponds to the mode of the ultrasound scanner.

Some embodiments may comprise: detecting the mode of the ultrasound scanner by detecting an activation of a button on the ultrasound scanner; detecting the mode of the ultrasound scanner by detecting a gesture of the ultrasound scanner; or detecting the mode of the ultrasound scanner by detecting an ultrasound scanning motion of the ultrasound scanner.

In some embodiments, the interface operates in: a navigation mode in which inputs to the ultrasound scanner cause navigation of elements displayed on the screen; or a scanning mode in which the ultrasound media is displayed on the screen.

Some embodiments may comprise: adjusting, by the ultrasound scanner, a depth of the ultrasound media displayed on the screen; adjusting, by the ultrasound scanner, a gain of the ultrasound media displayed on the screen; or freezing, by the ultrasound scanner, an image of the ultrasound media displayed on the screen.

Some embodiments may comprise: listening, by the interface, for the identification signal from the ultrasound scanner; by the interface, in response to detecting the identification signal, requesting to establish a communication link with the ultrasound scanner; participating, by the interface, in the establishing of the communication link; receiving, by the interface, the data; receiving, by the interface, the parameter; using, by the interface, the parameter to convert the data into an ultrasound media; and sending the ultrasound media to the screen for display thereon.

In some embodiments, the method may be performed by the ultrasound scanner and interface without human intervention, after the ultrasound scanner is switched on.

Some embodiments may comprise: receiving, by the interface, a signal indicative of a mode of the ultrasound scanner, wherein the mode is at least one of a navigation mode or a scanning mode; and the interface operating in a mode that corresponds to the mode of the ultrasound scanner.

Some embodiments may comprise the screen operating in: a navigation mode in which inputs to the ultrasound scanner cause navigation of elements displayed on screen; or a scanning mode in which the ultrasound media is displayed on the screen.

Some embodiments may comprise receiving, by the interface from the ultrasound scanner, a command to: adjust a depth of the ultrasound media displayed on the screen; adjust a gain of the ultrasound media displayed on the screen; or freeze an image of the ultrasound media displayed on the screen.

In another broad aspect of the present disclosure there is presented a method for establishing a wireless communication link between an ultrasound scanner and an interface that controls a screen, the method comprising: listening, by the interface, for an identification signal from the ultrasound scanner; by the interface, in response to detecting the identification signal, requesting to establish a communication link with the ultrasound scanner; by the interface, establishing a communication link to the ultrasound scanner; receiving, by the interface, data from an ultrasound scan; receiving, by the interface, a parameter; using, by the interface, the parameter to convert the data into an ultrasound media; and sending the ultrasound media to the screen for display thereon.

In some embodiments, the communication link is a Wi-Fi™ communication link or a Bluetooth™ communication link.

In some embodiments, the listening occurs when the screen is in a standby or hibernation state, the method comprising the interface switching on the screen before sending the ultrasound media to the screen.

Some embodiments comprise: receiving, by the interface, a signal indicative of a mode of the ultrasound scanner, wherein the mode is at least one of a navigation mode or a scanning mode; and the interface operating in a mode that corresponds to the mode of the ultrasound scanner.

Some embodiments comprise: the interface operating in: a navigation mode in which inputs to the ultrasound scanner are received by the interface and used by the interface to cause navigation of elements displayed on screen; or a scanning mode in which the ultrasound media is displayed on the screen.

Some embodiments comprise: receiving, by the interface from the ultrasound scanner, a command to: adjust a depth of the ultrasound media displayed on the screen; adjust a gain of the ultrasound media displayed on the screen; or freeze an image of the ultrasound media displayed on the screen; and causing the screen to alter the displayed ultrasound media according to the command.

In another broad aspect of the present disclosure there is presented a method for establishing a wireless communication link between an ultrasound scanner and an interface that controls a screen, the method comprising: listening, by the interface, for an identification signal from the ultrasound scanner; broadcasting, by the ultrasound scanner, an identification signal; by the interface, in response to detecting the identification signal, requesting to establish a communication link with the ultrasound scanner; detecting, by the ultrasound scanner, the request from the interface to establish a communication link with the ultrasound scanner; establishing, by the interface and the ultrasound scanner, the communication link; sending, by the ultrasound scanner to the interface, data from an ultrasound scan; receiving, by the interface, the data; sending, by the ultrasound scanner to the interface, a parameter; receiving, by the interface, the parameter; using, by the interface, the parameter to convert the data into an ultrasound media; and sending, by the interface, the ultrasound media to the screen for display thereon.

Some embodiments comprise the ultrasound scanner operating as a hotspot or using a Bluetooth™ communication protocol to broadcast the identification signal and establish the communication link.

Some embodiments are performed without human intervention after the ultrasound scanner is switched on.

Some embodiments comprise, initially: programming the ultrasound scanner to permit establishing the communication link; and programming the interface to recognize the identification signal before requesting establishment of the communication link; wherein a communication link is not established between the interface and another ultrasound scanner that broadcasts another identification signal that is not recognized by the interface.

In a further broad aspect of the present disclosure there is presented an ultrasound scanner that establishes a wireless communication link with an interface that controls a screen, comprising: a processor and computer readable memory storing computer readable instructions, which, when executed by the processer cause the ultrasound scanner to: broadcast an identification signal; detect a request from the interface to establish a communication link with the ultrasound scanner; establish the communication link; send, to the interface, data from an ultrasound scan; and send, to the interface, a parameter that is used by the interface to convert the data into an ultrasound media for display on the screen.

In some embodiments, the ultrasound scanner operates as a hotspot or using a Bluetooth™ communication protocol to broadcast the identification signal and establish the communication link.

In some embodiments, the computer readable instructions, when executed by the processor, cause the ultrasound scanner to: detect a mode of the ultrasound scanner, wherein the mode is at least one of a navigation mode or a scanning mode; and send, to the interface, a signal indicative of the mode; wherein the signal instructs the interface to operate in a mode that corresponds to the mode of the ultrasound scanner.

In some embodiments, the ultrasound scanner comprises a button, which, when activated, produces a signal that defines the mode of the ultrasound scanner.

In some embodiments, the ultrasound scanner comprises multi-axis motion sensor, wherein the computer readable instructions, when executed by the processor, cause the ultrasound scanner to detect the mode of the ultrasound scanner by detecting a gesture of the ultrasound scanner.

In some embodiments, the ultrasound scanner comprises a multi-axis motion sensor, wherein the computer readable instructions, when executed by the processor, cause the ultrasound scanner to detect that the mode of the ultrasound scanner is a scanning mode by detecting an ultrasound scanning motion of the ultrasound scanner.

In some embodiments, the ultrasound scanner comprises a button, which, when activated, causes a command to be transmitted by the ultrasound scanner to the interface that: adjusts a depth of the ultrasound media displayed on the screen; adjusts a gain of the ultrasound media displayed on the screen; or freezes, an image of the ultrasound media displayed on the screen.

In a further broad aspect of the present disclosure there is presented an interface that controls a screen and establishes a wireless communication link with an ultrasound scanner, comprising: a processor and computer readable memory storing computer readable instructions, which, when executed by the processer cause the interface to: listen for an identification signal from the ultrasound scanner; in response to detecting the identification signal, request establishment of a communication link with the ultrasound scanner; establish the communication link with the ultrasound scanner; receive data from the ultrasound scanner; receive a parameter from the ultrasound scanner; use the parameter to convert the data into an ultrasound media; and send the ultrasound media to the screen for display thereon.

In a further broad aspect of the present disclosure there is presented an ultrasound scanning system comprising: an ultrasound scanner and an interface that controls a screen. The ultrasound scanner comprises: a processor; computer readable memory storing computer readable instructions, which, when executed by the processer cause the ultrasound scanner to: broadcast an identification signal; detect a request from the interface to establish a communication link with the ultrasound scanner; establish the communication link; send, to the interface, data from an ultrasound scan; and send, to the interface, a parameter. The interface comprises: a further processor; further computer readable memory storing computer readable instructions, which, when executed by the further processer cause the interface to: listen for the identification signal from the ultrasound scanner; in response to detecting the identification signal, request establishment of the communication link with the ultrasound scanner; establish the communication link with the ultrasound scanner; receive the data from the ultrasound scanner; receive the parameter from the ultrasound scanner; use the parameter to convert the data into an ultrasound media; and send the ultrasound media to the screen for display thereon.

In some embodiments, the interface is removably connectable to the screen.

In some embodiments, the interface is integral with the screen.

1. A method for establishing a wireless communication link between an ultrasound scanner and an interface that controls a screen, the method comprising:
broadcasting, by the ultrasound scanner, an identification signal;
detecting, by the ultrasound scanner, a request from the interface to establish a communication link with the ultrasound scanner;
establishing, by the ultrasound scanner, the communication link;
sending, by the ultrasound scanner to the interface, data from an ultrasound scan; and
sending, by the ultrasound scanner to the interface, a parameter that is used by the interface to convert the data into an ultrasound media for display on the screen.

2. The method of claim 1, comprising the ultrasound scanner operating as a Wi-Fi™ hotspot or using a Bluetooth™ communication protocol to broadcast the identification signal and establish the communication link.

3. The method of claim 1, performed by the ultrasound scanner without human intervention, after the ultrasound scanner is switched on.

4. The method of claim 1, comprising:
detecting, by the ultrasound scanner, a mode of the ultrasound scanner, wherein the mode is at least one of a navigation mode or a scanning mode; and
sending, by the ultrasound scanner to the interface, a signal indicative of the mode;
wherein the signal instructs the interface to operate in a mode that corresponds to the mode of the ultrasound scanner.

5. The method of claim 4, comprising:
detecting the mode of the ultrasound scanner by detecting an activation of a button on the ultrasound scanner.

6. The method of claim 4, comprising:
detecting the mode of the ultrasound scanner by detecting a gesture of the ultrasound scanner.

7. The method of claim 4, comprising:
detecting the mode of the ultrasound scanner by detecting an ultrasound scanning motion of the ultrasound scanner.

8. The method of claim 1, comprising:
adjusting, by the ultrasound scanner, a depth of the ultrasound media displayed on the screen;
adjusting, by the ultrasound scanner, a gain of the ultrasound media displayed on the screen; or
freezing, by the ultrasound scanner, an image of the ultrasound media displayed on the screen.

9. The method of claim 1, comprising:
listening, by the interface, for the identification signal from the ultrasound scanner;
by the interface, in response to detecting the identification signal, requesting to establish a communication link with the ultrasound scanner;
participating, by the interface, in the establishing of the communication link;
receiving, by the interface, the data;
receiving, by the interface, the parameter;
using, by the interface, the parameter to convert the data into an ultrasound media; and
sending the ultrasound media to the screen for display thereon.

10. The method of claim 9, comprising the interface:
receiving a signal indicative of a mode of the ultrasound scanner, wherein the mode is at least one of a navigation mode or a scanning mode; and
operating in a mode that corresponds to the mode of the ultrasound scanner;
wherein, in the navigation mode, inputs to the ultrasound scanner cause navigation of elements displayed on the screen;
wherein, in the scanning mode, the ultrasound media is displayed on the screen.

11. A method for establishing a wireless communication link between an ultrasound scanner and an interface that controls a screen, the method comprising:
listening, by the interface, for an identification signal from the ultrasound scanner;
by the interface, in response to detecting the identification signal, requesting to establish a communication link with the ultrasound scanner;
by the interface, establishing a communication link to the ultrasound scanner;
receiving, by the interface, data from an ultrasound scan;
receiving, by the interface, a parameter;
using, by the interface, the parameter to convert the data into an ultrasound media; and
sending the ultrasound media from the interface to the screen for display thereon.

12. The method of claim 11, wherein the listening occurs when the screen is in a standby or hibernation state, the method comprising the interface switching on the screen before sending the ultrasound media to the screen.

13. The method of claim 11, comprising:
receiving, by the interface, a signal indicative of a mode of the ultrasound scanner, wherein the mode is at least one of a navigation mode or a scanning mode; and
the interface operating in a mode that corresponds to the mode of the ultrasound scanner;
wherein, in the navigation mode, inputs to the ultrasound scanner cause navigation of elements displayed on screen;
wherein, in the scanning mode, the ultrasound media is displayed on the screen.

14. The method of claim 11, comprising:
receiving, by the interface from the ultrasound scanner, a command to:
adjust a depth of the ultrasound media displayed on the screen;
adjust a gain of the ultrasound media displayed on the screen; or
freeze an image of the ultrasound media displayed on the screen; and
causing the screen to alter the displayed ultrasound media according to the command.

15. An ultrasound scanner that establishes a wireless communication link with an interface that controls a screen, comprising:
a processor; and
computer readable memory storing computer readable instructions, which, when executed by the processor cause the ultrasound scanner to:
broadcast an identification signal;
detect a request from the interface to establish a communication link with the ultrasound scanner;
establish the communication link;
send, to the interface, data from an ultrasound scan; and
send, to the interface, a parameter that is used by the interface to convert the data into an ultrasound media for display on the screen.

16. The ultrasound scanner of claim 15, wherein the computer readable instructions, when executed by the processor, cause the ultrasound scanner to:
detect a mode of the ultrasound scanner, wherein the mode is at least one of a navigation mode or a scanning mode; and
send, to the interface, a signal indicative of the mode;
wherein the signal instructs the interface to operate in a mode that corresponds to the mode of the ultrasound scanner.

17. The ultrasound scanner of claim 16, comprising a button, which, when activated, produces a signal that defines the mode of the ultrasound scanner.

18. The ultrasound scanner of claim 16, comprising a multi-axis motion sensor, wherein the computer readable instructions, when executed by the processor, cause the ultrasound scanner to:
detect the mode of the ultrasound scanner by detecting a gesture of the ultrasound scanner.

19. The ultrasound scanner of claim 16, comprising a multi-axis motion sensor, wherein the computer readable instructions, when executed by the processor, cause the ultrasound scanner to:
detect that the mode of the ultrasound scanner is a scanning mode by detecting an ultrasound scanning motion of the ultrasound scanner.

20. The ultrasound scanner of claim 16, comprising a button, which, when activated, causes a command to be transmitted by the ultrasound scanner to the interface that:
adjusts a depth of the ultrasound media displayed on the screen;
adjusts a gain of the ultrasound media displayed on the screen; or
freezes, an image of the ultrasound media displayed on the screen.
